(19) United States
(12) Patent Application Publication
(10) Pub. No.: US 2021/0398372 A1
(43) Pub. Date: Dec. 23, 2021

(54) METHODS AND APPARATUS FOR SECURE STERILIZATION OF INDIVIDUAL ASSETS USING UV-C RADIATION

(71) Applicant: 1 Micro, LLC, Waconia, MN (US)

(72) Inventor: Jon Kristian Terp, Waconia, MN (US)

(21) Appl. No.: 16/906,957

(22) Filed: Jun. 19, 2020

(51) Int. Cl. G07C 9/00; G16H 40/20; G06K 7/14; G06K 7/10

(52) U.S. Cl. CPC G07C 9/00714; G06K 7/10297; G06K 7/1417; G16H 40/20

(57) ABSTRACT

A secure sterilization system comprises at least a plurality of sterilization compartments contained within a device, and a control system for granting individual users access to unique ones of the secure compartments. The plurality of sterilization compartments include circuitry and components that include UV-C LEDs and UV-C photodetectors. In some embodiments, a plurality of walls within the sterilization compartment include circuitry and components that include UV-C LEDs and UV-C photodetectors. In some embodiments, a plurality of walls within a sterilization compartment include sensor components for presence/absence detection of assets within a compartment. Administrator-controlled versions of a secure sterilization system include access drawers with multiple sterilization compartments.

### TECHNICAL FIELD

Embodiments relate generally to sterilization and disinfection. More particularly, embodiments relate to the use of UV-C LEDs within a closed, secure compartment to sterilize or disinfect individual assets, particularly personal, medical or surgical protective equipment.

### BACKGROUND OF THE INVENTION

It is well-established that ultraviolet (UV) radiation is effective in killing microorganisms including but not limited to bacteria, viruses, yeasts and molds. UV radiation is widely used for sanitizing and disinfecting surfaces in various industries including healthcare, food processing, research laboratories, air purification systems and water purification applications. Ultraviolet radiation/light is electromagnetic radiation having a wavelength ranging from approximately 10 nanometers (nm) to approximately 400 nanometers. Ultraviolet-C (UV-C) is a range of electromagnetic radiation having a wavelength ranging from approximately 100 nanometers to approximately 280 nanometers. UV-C light in particular has demonstrated to be up to 99.9% effective in the sanitization of microorganisms and biological pollutants/pathogens such as viruses, bacteria, molds, dust mites and flea eggs.

There are several advantages to utilizing UV-C light, in addition to the effectiveness described above. UV-C light requires only electricity, and there are no potentially hazardous chemicals and the associated storage challenges presented thereby. UV-C light leaves no residue, does not require drying time, cannot spill or splash, requires minimal skill and/or effort on the part of the operator, and uses long-lasting bulbs that require very little inventory management.

Safely using UV-C light to disinfect surfaces does present some unique problems. First, UV-C light sources cast shadows. Areas in shadows may not be properly disinfected. Second, UV-C light bulbs, like nearly all light bulbs, are relatively fragile and present dangers if broken. Third, UV-C radiation is harmful to humans, especially in high-intensity applications like those used in disinfecting procedures. These issues have been addressed in various ways in commercial, manufacturing and high-volume contexts. Approaches for identifying and tracking items to be disinfected by a UV light sources are described, for example, in U.S. Pat. Nos. 9,056,147, 9,542,663, and 10,255,466 have included the use of bar codes and RFID tags associated with the item to aid in identifying and tracking individual items. Other approaches have used sensors or identification codes to determine the presence or absence of an item in a disinfection device as described, for example, in U.S. Pat. Nos. 9,057,657 and 9,672,726. While these approaches may be useful in the context of batch decontamination processing, these approaches may not be suitable in the context of quick and simple disinfection of individual items, especially where the individual user is one of a multitude of users who want to utilize a disinfection process.

A particular problem associated with the limitations of UV-C sterilization in the context of sterilizing individual assets of different users is the difficulty of associating identifiers with different individual items to be sterilized as this can lead to cross-contamination of personal protective equipment (PPE) and other individual assets. In addition to the added complexity and components that need to be added to the individual asset, such identifiers may not be suitable for attachment to an asset or may be destroyed or damaged during the sterilization process. There is a need for a UV-C disinfection and sterilization system that exploits the advantages of UV-C light, while also addressing the aforementioned problems in the context of sterilization of a small number of individual assets used by different users.

### SUMMARY OF THE INVENTION

A secure sterilization system in accordance with various embodiments comprises at least a plurality of sterilization compartments contained within a device, a connection to a database that contains user information and asset information obtained from machine-readable media, a media input device for performing analysis of machine-readable media, and a control system for granting user access to secure compartments. In various embodiments, the secure sterilization system is configured for secure transfer of PPE or other individual sterile items from a user to a particular individual compartment of a secure sterilizer unit and back to the user without the need for individual identifiers of each item and without cross-contamination or inadvertent exchange of individual items.

Prior art asset access systems that provide sterilization of individual assets, such as personal protective equipment (PPE) of a given individual do not have the potential to be utilized extensively in systems where the individual asset may not have individual identification or the owner of the asset does not have a particular human involved in the transaction. In various embodiments, there is no need for dedicated hardware on the part of the user that must be attached to an individual asset for identification will permit the widespread deployment of these asset access sterilization systems. In various embodiments, a non-hardware-specific system that allows users to gain access to individual PPE sterilization assets and complete transactions for these asset management and access systems. One of the advantages of such a sterilization access system is to allow individual users to have their own PPE assets, for example, sterilized and reused without another human involved contact and without the possibility of mixing up an asset of the given individual with an asset of another individual.

In embodiments, the plurality of sterilization compartments include circuitry and components that include UV-C LEDs and UV-C photodetectors. In some embodiments, a plurality of walls within the sterilization compartment include circuitry and components that include UV-C LEDs and UV-C photodetectors. In some embodiments, a plurality of walls within a sterilization compartment include components for presence/absence detection of assets within a compartment.

A secure sterilization system in accordance with various embodiments comprises at least a plurality of sterilization drawers, each drawer with a plurality of sterilization compartments contained within a device, a connection to a database that contains user information, administrator information and asset information obtained from machine-readable media, a media input device for performing analysis of machine-readable media, and a control system for granting user access to secure compartments.

The control system in accordance with various embodiments utilizes a two-dimensional (2D) code associated with a particular drawer or door that allows a user to gain physical access to and insert an item to be sterilized. Users of the system will present the 2D code to a transaction client that grants the user access to a sterilization drawer. Transaction clients can reside at various facilities that do not employ humans as part of the asset transfer to the user. Automated transaction clients can reside at facilities that include, but are not limited to, hospitals, research and testing laboratories, vehicle parking facilities, storage and rental facilities, bank ATMs, vending machines, school, hospital or gym lockers, luggage handling locations such as airports or train stations, and hotel room doors. Upon completion of the sterilization of the item, the user will present a new 2D code to the human-less access system that allows the user to retrieve the sterilized item from the sterilization drawer at the transaction client.

In embodiments, the plurality of sterilization compartments within the plurality of sterilization drawers include circuitry and components that include UV-C LEDs and UV-C photodetectors. In some embodiments, a plurality of walls within the plurality of sterilization compartments within the plurality of sterilization drawers include circuitry and components that include UV-C LEDs and UV-C photodetectors. In some embodiments, a plurality of walls within the plurality of sterilization compartments within the plurality of sterilization drawers include sensor components for presence/absence detection of assets within a compartment.

### BRIEF DESCRIPTION OF THE DRAWINGS

FIG. 1 illustrates an incorporated by reference remote key fob access system transacted with a mobile communication device.

FIG. 2 illustrates an individual PPE asset to be sterilized in the form of a face mask.

FIG. 3 illustrates an embodiment of a PPE sterilizing device.

FIG. 4 illustrates embodiments of a sterilization compartment for a PPE sterilizing device.

FIG. 5 illustrates a functional flow chart of an asset insertion/extraction control system for a PPE sterilizing device.

FIG. 6 illustrates a functional flow chart of asset insertion process of an asset insertion/extraction control system for a PPE sterilizing device.

FIG. 7 illustrates a functional flow chart of asset extraction process of an asset insertion/extraction control system for a PPE sterilizing device.

FIG. 8 illustrates an embodiment of a PPE sterilizing device.

### DETAILED DESCRIPTION OF THE DRAWINGS

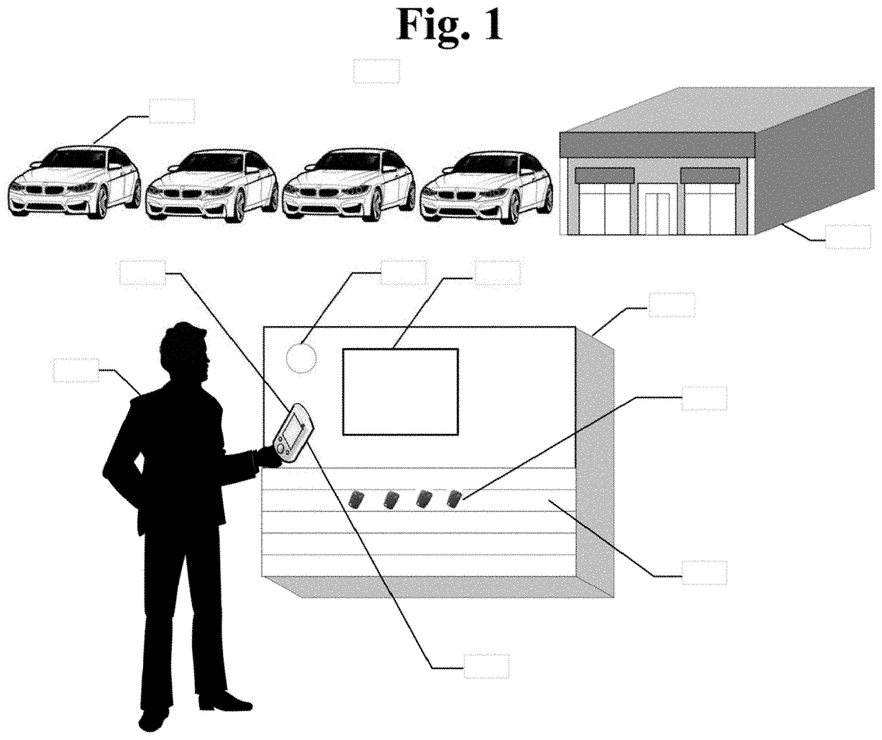

Remote access to physical assets occurs in many forms today. In embodiments, secure access to sterilized assets is enabled. Embodiments of compartment-based secure access systems are disclosed in U.S. Pat. No. 10,665,047 (Terp) which is hereby incorporated by reference in its entirety. FIG. 1 illustrates a remote key fob access system transacted with a mobile communication device.

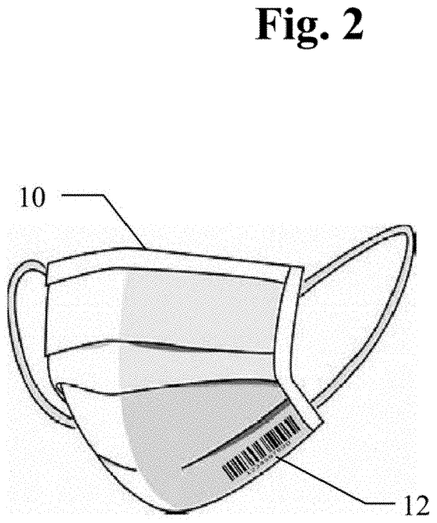

FIG. 2 illustrates an individual asset to be sterilized in the form of a face mask 10 that optionally includes a code 12 that can be scanned or analyzed by a camera. In embodiments, the asset 10 presented for sterilization in the system may include, but not be limited to, medical or surgical protective equipment, medical, surgical or dental instruments, and personal protective equipment (PPE). Code 12 may be a unique identifier for each individual or type of item to be sterilized by the system. A code can remain in effect for the life of an asset or new code IDs can be established in order to have a unique code ID for each item for each transaction. The data associated with a code may include an alphanumeric description of the item and may contain many sub-fields for information that may include, but not be limited to, an associated sanitation protocol, such as time or intensity for UV-C exposure, make, materials, a record of lifetime use/sanitation cycles, expiration date, total number of sanitation cycles per lifetime.

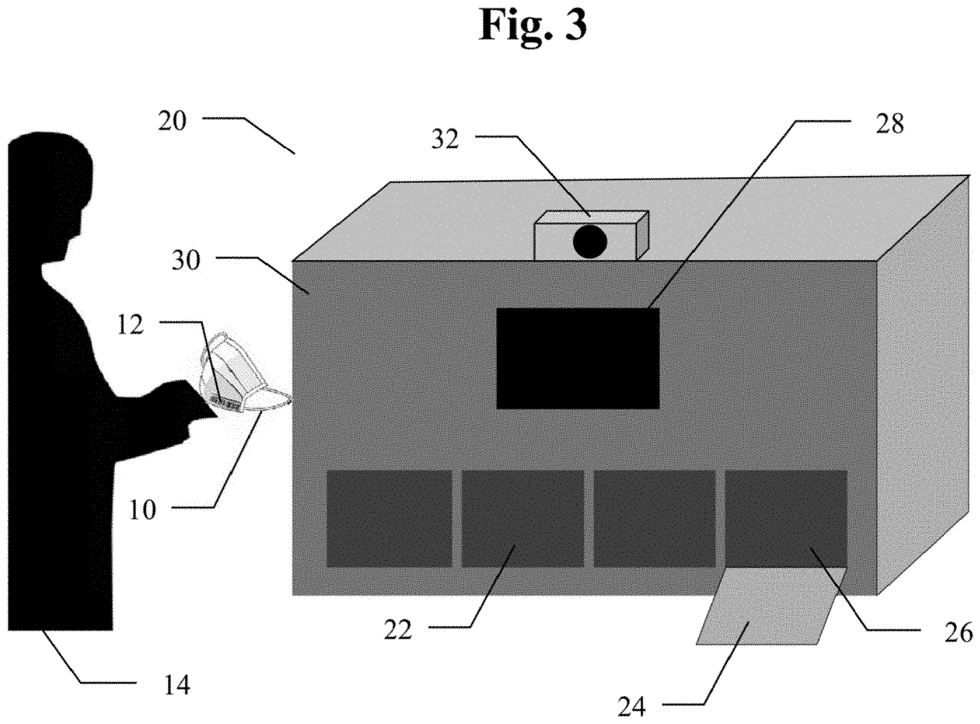

FIG. 3 illustrates an embodiment of a PPE sterilizing device 20 with a plurality of closed access doors 22. An open access door 24 is shown with an exposed sterilization compartment 26. In embodiments, a display 28 is included as part of or integrated into a front panel 30. In embodiments, a camera 32 is operably connected to the sterilizing device 20 for imaging and analyzing asset 10 identification 12 for purposes of secure asset 10 insertion to the device 20. In embodiments, a camera 32 is operably connected to the sterilizing device 20 for imaging and analyzing user 14 identification for purposes of secure asset 10 extraction from the device 20.

User 14 may be identified by a static identifier, such as an identification badge or facial recognition, or a dynamic identifier, such as a randomized numeric, bar, or QR code.

For example, in an on-site transaction with a system in order to insert an item to be sterilized, the user 14 may present a 2D code to the system by showing the screen of a mobile device to the optical unit input, such as camera 32, of the system. The asset sanitation system may contain multiple zones 22 that have covers or other actuated features to secure an item during sanitation and until retrieval by user 14. The screen 28 or other passive feedback device displays messages that communicate transaction details to the user 14. The transaction of the user 14 with the sanitation system does not require the presence of any personnel.

Upon completion of the presentation by the user 14 of the 2D code on the screen of the mobile communication device to the optical input unit 32, and upon the subsequent validation of the 2D code, the system will actuate opening of a sterilization compartment, e.g., compartment 26. Upon the sensed insertion of the asset 12 the system will display the appropriate status message at the display 28 and complete the transaction by closing the compartment 26 that contains the asset to be sanitized.

The system of FIG. 3 also facilitates the return of a physical asset 12 at the end of a sanitation cycle, or may secure the item for longer if the user 14 is not ready to retrieve the item 12 immediately once sanitize. The user 14 will present the appropriate 2D code displayed on a mobile device to the optical input unit 32 as a means to initiate the return transaction. The 2D code may be the same code presented upon insertion of the item 12, or a refreshed or otherwise updated code. Upon validation of the 2D code, the system will actuate the appropriate access zone 24 to enable the user's 14 removal of the sanitized item from the compartment 26. The return system will display 28 the successful completion of the return transaction and close the actuator for the access zone 24.

Embodiments substantially embodying the individual access exemplified in FIG. 3 may be particularly advantageous in closed environments or high traffic settings. For example, an individual access embodiment could enable travelers to sanitize luggage before or after boarding an aircraft, a train, a bus, etc. The settings of the UV-C exposure, such as intensity and duration, may be determined according to the environment the device is located. For instance, to continue the public transportation example, a very high intensity exposure may be desirable to allow for short duration and quick turnover for traveler convenience.

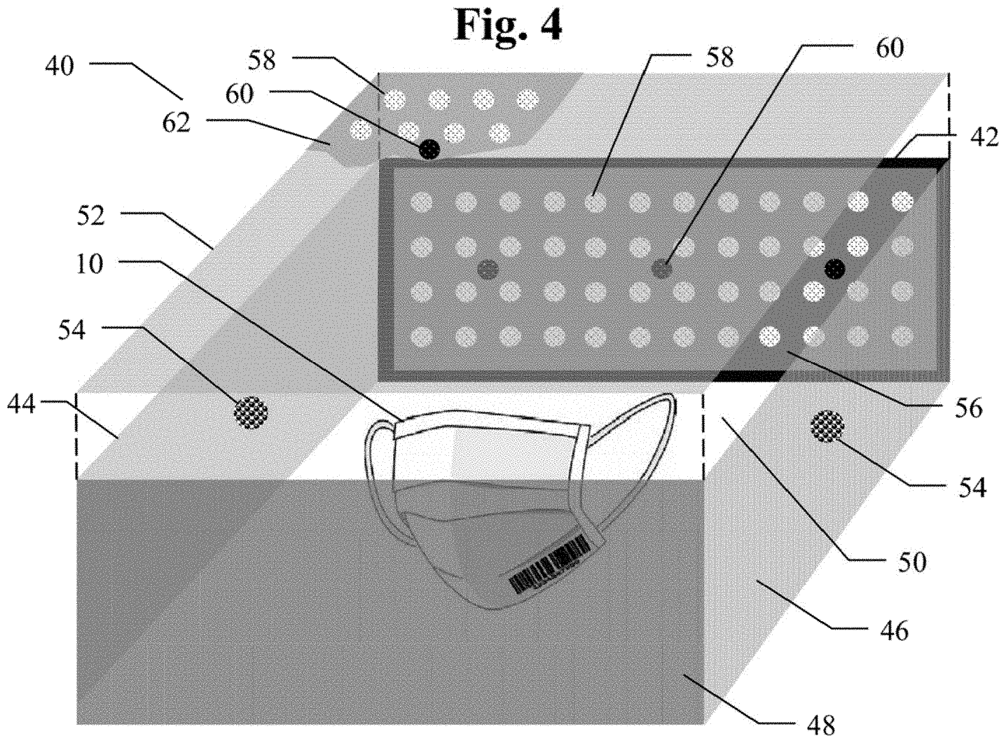

FIG. 4 illustrates embodiments of a sterilization compartment 40 for a PPE sterilizing device used for the safe and secure sterilization of an asset 10. In embodiments, each compartment 40 is constructed with a back wall 42, a left wall 44, a right wall 46, a front wall 48 in the form of an as-shown closed access door, a compartment base 50 and a compartment ceiling 52. In embodiments, active sensing presence/absence detectors 54 are affixed to or embedded into the left wall 44 and the right wall 46. In embodiments, affixed to the back wall 42 is a circuit board 56 that includes a plurality of UV-C LED 58 components and a plurality of UV-C photodetector 60 elements. In embodiments, affixed to the compartment ceiling 52 is a circuit board 62 that includes a plurality of UV-C LED 58 components and a plurality of UV-C photodetector 60 elements.

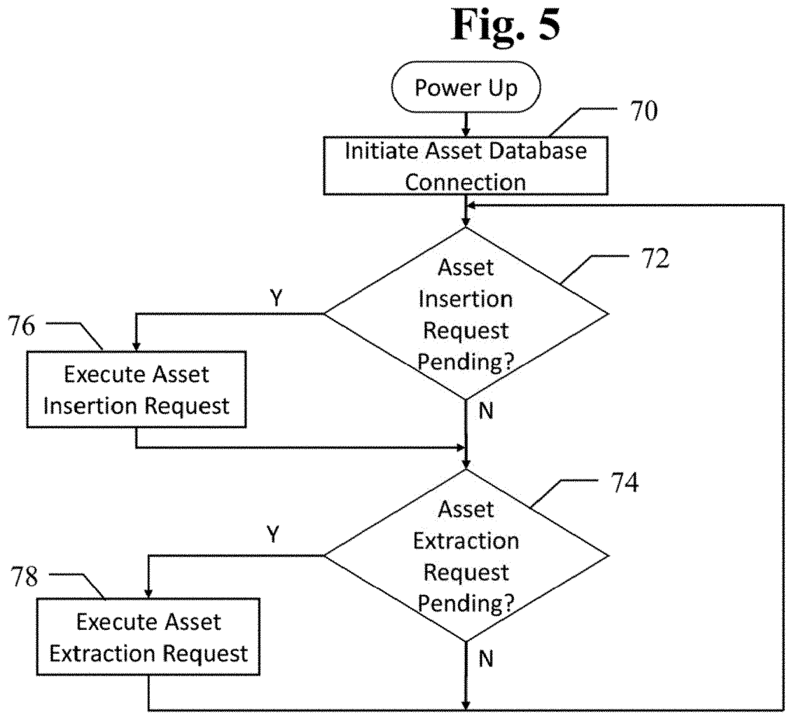

FIG. 5 illustrates a functional flow chart of an asset insertion/extraction control system for a PPE sterilizing device. In embodiments, access to a device is granted by the control system working in concert with an access database. Therefore, upon power up, the control system will initiate 70 the connection to an asset database that stores the identification information for all authorized users and optionally for all authorized assets. In embodiments, by way of interaction with the device camera interface, the control system checks for a pending insertion request 72. Upon validation of a valid insertion request, the control system executes an asset insertion request 76. In embodiments, the control system checks for a pending extraction request 74. Upon validation of a valid extraction request, the control system executes an asset extraction request 78. In some embodiments, one or both of the insertion request and the extraction request are facilitated by way of interaction with the device camera interface.

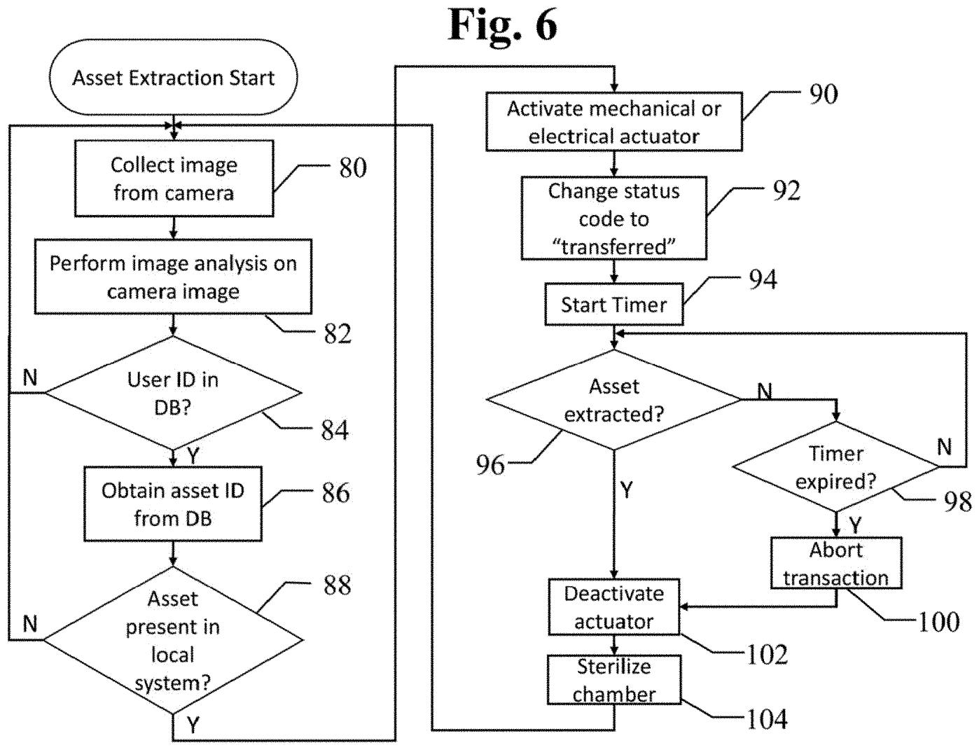

FIG. 6 illustrates a functional flow chart for an asset extraction process. Upon the control system prompting a user to identify themselves, a user will present identification to the camera in the form of an optically analyzed badge or card that contains a code that resides in an access database. In some embodiments, a camera will collect 80 an image and perform analysis 82 on the acquired image. If the user ID 84 is not in the database, the process returns to collect another image 80. If the user ID 84 exists in the database, the control system performs a database query for the associated asset ID 86 and determines if the asset is present in the local system 88. If the asset is not in the local system, the process returns to collect another image 80. If the asset is in the local system, the process will activate 90 the electrical or mechanical actuator for the proper access door, thus allowing the user to extract the sterilized asset. The status code of the asset is changed to “transferred” 92 and a timer is started 94 that is used to monitor the elapsed time to extract the asset. A test for a successfully extracted asset 96 involves monitoring the presence/absence detectors (such as detectors 54 is FIG. 4) within the selected sterilization compartment. Upon sensing that the asset has been removed, the actuator is deactivated 102 and the compartment is sterilized 104. While waiting for successful extraction of an asset, the control system monitors a system timer 98 and upon expiration of the timer will abort 100 the transaction.

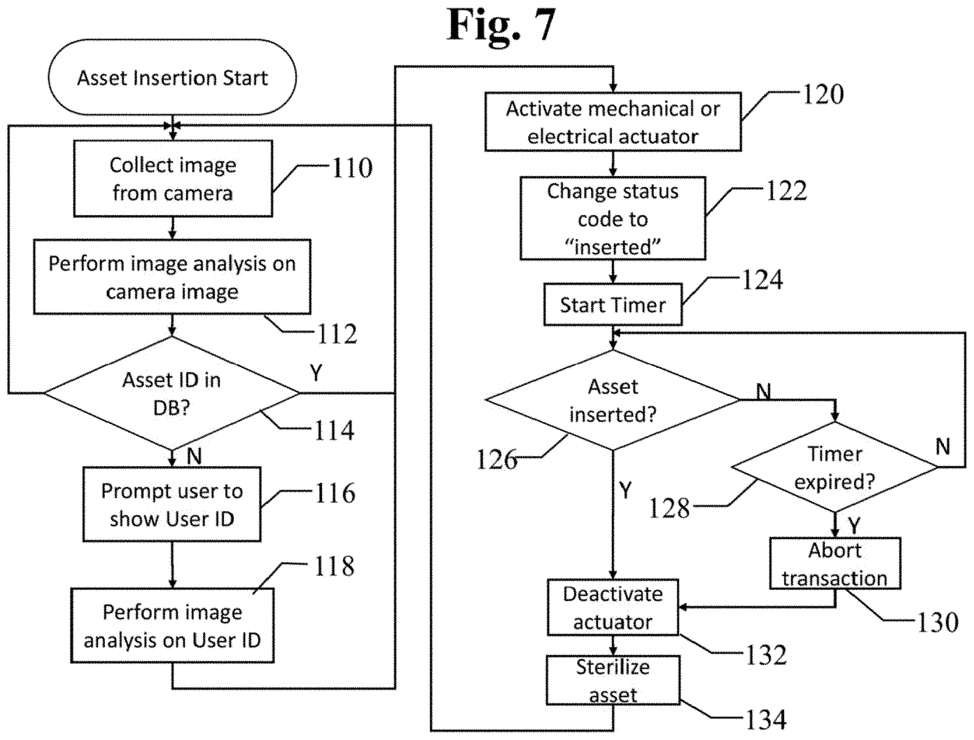

FIG. 7 illustrates a functional flow chart for an asset insertion process. Upon the control system prompting a user to identify an asset either by way of an app on mobile device of the user, or by a user presenting to the camera an asset that includes an optically analyzed identification code or sequence. A camera will collect 110 an image and perform analysis 112 on the acquired image. If the asset ID 114 is not in the database, the process returns to collect another image 110. If the asset ID 114 exists in the database, the control system prompts the user to show their user ID 116 and the system performs image analysis 118 on the user ID. It is the analyzed user ID that will be used to allow user access to the sterilized asset during a subsequent extraction. If the asset is in the local system, the process will activate 120 the electrical or mechanical actuator for the proper access door, thus allowing the user to insert the asset for sterilization. The status code of the asset is changed to “inserted” 122 and a timer is started 124 that is used to monitor the elapsed time to insert the asset. A test for a successfully inserted asset 116 involves monitoring the presence/absence detectors within the selected sterilization compartment. Upon sensing that the asset has been inserted, the actuator is deactivated 132 and the compartment content is sterilized 134. While waiting for successful insertion of an asset, the control system monitors a system timer 128 and upon expiration of the timer will abort 130 the transaction.

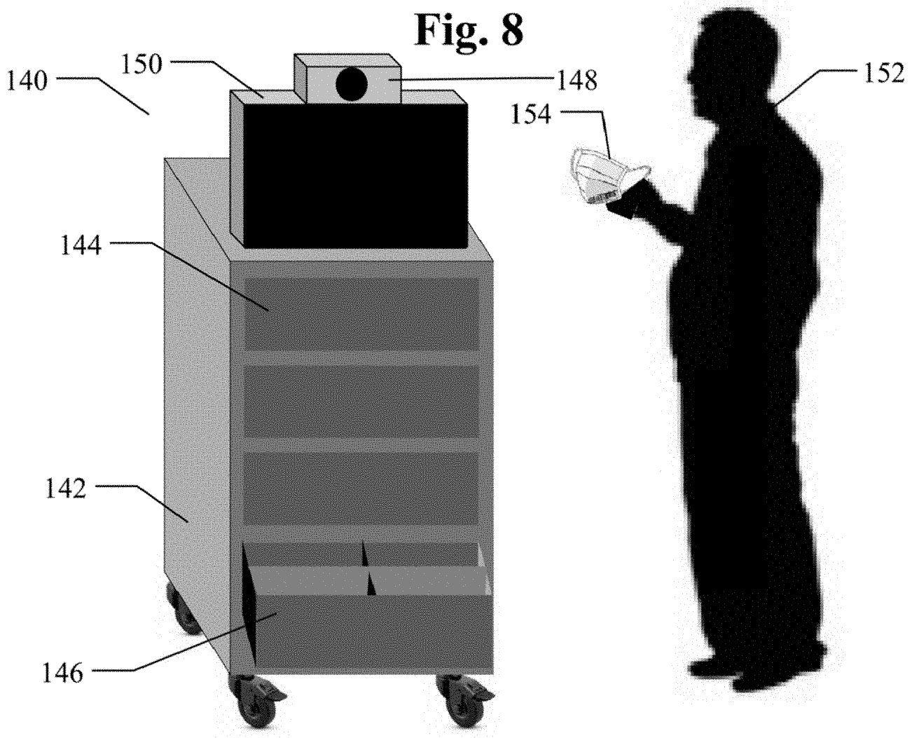

FIG. 8 illustrates an embodiment of an administrator-controlled PPE sterilizing device 140 with a plurality of closed access drawers 144. An open access drawer 146 is shown with a plurality of exposed sterilization compartments. In embodiments, a display 150 is included as part of or mounted on a chassis 142. In some embodiments, a camera 148 is operably connected to the sterilizing device 140 for imaging and analyzing asset 154 identification for purposes of secure asset 154 insertion to the device 140. In some embodiments, a camera 148 is operably connected to the sterilizing device 140 for imaging and analyzing administrator 152 identification for purposes of secure asset 154 extraction from the device 140.

Embodiments substantially embodying example device 140 may be advantageously used by personnel running a checkpoint or other centralized control of items to be sanitized. For example, in a manufacturing setting, a crew or shift may all need to return PPE or other items for sanitation at the same time, or to assist customs agents from minimizing introduction of contaminants by sanitizing the luggage of international travelers. Embodiments similar to example device 140 may also be advantageously used in clean settings where each individual may have multiple pieces of PPE to sanitized at one time, such that an individual may have sole access to a drawer 146 and each PPE item may be placed in a separate compartment to avoid shadowing and other interference between items.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. An access system configured to enable a transaction in which a user gains selective access to one of a plurality of secure sterilization compartments, the access system comprising:
a housing having one or more actuated features configured to provide selective access to sterilize one or more individual assets within one of the plurality of secure sterilization compartments within the housing having at least one UV-C LED selectively operable within each secure sterilization compartment, the housing including—
an access unit configured to receive a unique code presented by the user or a unique code associated with one of the plurality of secure sterilization compartments;
a computing platform configured to (i) validate the unique code received by the access unit, (ii) generate an asset ID code identifying at least one of the one or more assets to be sterilized within the housing, (iii) actuate one of the secure sterilization compartments corresponding to the asset ID code, thereby permitting selective access to the one of the secure sterilization compartments for the user insertion or retrieval of the one or more individual assets to be sterilized, and (iv) control a timing and an operation of the at least one UV-C LEDs associated the one of the secure sterilization compartments to sterilize the one or more individual assets associated with the asset ID code; and
a sensor configured with each secure sterilization compartment to sense a retrieval or an insertion of the one or more individual assets for that secure sterilization compartment, wherein the computing platform is further configured to update the asset ID code of the one more individual asset to reflect a change in status based on the retrieval or the insertion of the one or more individual assets.

2. The access system of claim 1, wherein each secure sterilization compartment includes at least one of a door or a drawer selectively controlled by the computing platform.

3. The access system of claim 1, wherein each secure sterilization compartment defines a volume of less than 1 cubic meter.

4. The access system of claim 1, wherein the timing and operation of the at least one-UV-C LED is configured to sterilize the one or more individual assets within the corresponding secure sterilization compartment in less than two minutes.

5. The access system of claim 1, wherein the timing and operation of the at least one-UV-C LED is configured to sterilize the one or more individual assets within the corresponding secure sterilization compartment in less than 30 seconds.

6. The access system of claim 1, further comprising at least one UV-C photodetector configured to determine an intensity of light from the UV-C LEDs and operably coupled to the computing platform to be utilized to manage the timing and the operation of the at least one UV-C LEDs.

7. The access system of claim 6, wherein the compartment comprises one or more walls that house circuitry and components that include the UV-C LEDs and the UV-C photodetectors.

8. The access system of claim 1 wherein the access unit is an optical unit configured to receive a unique code presented by the user or a unique code associated with associated with one of the plurality of secure sterilization compartments.

9. The access system of claim 8, wherein the unique code is a dimensional (2D) code selected from one of the following kinds of codes:
QR code, Aztec Code, Code 1, Color Code, Color Construct Code, CrontoSign, CyberCode, DataGlyphs, Data Matrix, Datastrip Code, digital paper, EZcode, High Capacity Color Barcode, Han Xin Barcode, HueCode, InterCode, MaxiCode, MMCC, NexCode, PDF417, Qode, Shot Code, Snap Code, SparQ Code, Voice Eye, or RFID.

10. The access system of claim 1 wherein the access unit is an RFID reader configured to receive the unique code presented by an RFID tag associated with the user or with one of the plurality of secure sterilization compartments.

11. The access system of claim 1 wherein the access unit is an app executable on a mobile device of the user configured to transmit the unique code presented by the user or the unique code associated with one of the plurality of secure sterilization compartments.

12. The access system of claim 7 wherein the app is configured to display details of the transaction to the user.

13. The access system of claim 1 wherein the access system further comprises a display screen configured to display details of the transaction.

14. The access system of claim 1, wherein the unique code includes information relating to both the user and the one or more individual assets.

15. The access system of claim 1, the computer processing system further comprises an administration program by which the access system enables an administrator to access multiple ones of the secure sterilization compartments.
